(19) United States
(12) Patent Application Publication
(10) Pub. No.: US 2021/0113786 A1
(43) Pub. Date: Apr. 22, 2021

(54) INHALER DEVICE FOR INHALABLE LIQUIDS

(71) Applicant: Medical Developments International Limited, Scoresby (AU)

(72) Inventors: Greg Rowland, Scoresby (AU); Jake Golding, Scoresby (AU); Edward Linacre, Abbotsford (AU); Viktor Legin, Abbotsford (AU)

(21) Appl. No.: 17/138,132

(22) Filed: Dec. 30, 2020

Related U.S. Application Data
(63) Continuation of application No. 15/745,871, filed on Jan. 18, 2018.

(30) Foreign Application Priority Data
Jul. 20, 2015 (AU) 2015902866

(51) Int. Cl. A61M 11/04; A61M 15/06; A61M 15/00; A61P 29/00; A61K 9/00; A61K 31/08

(52) U.S. Cl. CPC A61M 11/04; A61M 15/06; A61M 15/0036; A61M 15/002; A61M 16/01; A61P 29/00; A61K 9/0078; A61K 31/08; A61M 15/0043

(57) ABSTRACT

An inhaler device includes a sealed elongated body having air inlet and mouthpiece ends, a first end-seal sealing the air inlet end, a second end-seal sealing the mouthpiece end, and passive evaporation support material pre-loaded with methoxyflurane liquid. As the methoxyflurane liquid forms a vapor, the elongated body forms a vapor chamber such that the stored vapor is available for direct administration upon opening the first and second end-seals. The first end-seal is a resealable, adjustable end cap with an air inlet groove or hole that forms an air inlet opening when opened such that the air inlet's size is adjustable. The second end-seal is a resealable, adjustable end cap including a mouthpiece chamber configured such that when closed the vapor chamber is sealed from the mouthpiece chamber and when open vapor flows radially inward into the mouthpiece chamber through vents located in a side wall of the mouthpiece chamber.

### CROSS-REFERENCE TO RELATED APPLICATION(S)

This application is a continuation of application Ser. No. 15/745,871, filed Jan. 18, 2018, which claims priority of International Application No. PCT/AU2016/050637, filed Jul. 19, 2016, and published as WO/2017/011866A1 on Jan. 26, 2017, in English, which claims priority to Australian patent application 2015902866, filed Jul. 20, 2015, in English, the contents of which are each hereby incorporated by reference in their entirety.

### FIELD

The present invention relates to an inhaler device for inhalable liquids, in particular for the storage and/or administration of inhalable volatile liquids such as halogenated volatile liquids, to a patient.

### BACKGROUND

The storage and administration of inhalable liquids to patients that comprise active agents, or that are themselves the active agent, commonly presents challenges. Due to patient preference and ease of self-administration or administration in a hospital setting or other settings as required, active agents such as therapeutic agents or pharmaceutical agents, are often formulated for oral delivery in the form of tablets and capsules, nasal delivery in the form of sprays and liquid formulations for intravenous delivery.

Where it is advantageous to administer active agents to a patient's lungs, for example to treat or alleviate respiratory diseases, the active agent may be administered by the oral inhalation route, alone or in combination with the intranasal route. Suitable inhaler devices may include, for example, metered dose inhalers and dry powder inhalers. These types of oral inhalation devices typically require pressurised means to deliver the active agent to the desired site of action in the lungs. In addition, liquids that contain active agents or that are themselves the active agent usually require transformation into an inhalable, respirational, form at the point of administration to be suitable for delivery by the inhalation route. Transforming a liquid into an inhalable form, such as by nebulisation or aerosolizing into respirational sized droplets or heating to form a vapour, requires delivery devices to include moving, mechanical, heating and/or electrical means which adds to the complexity of the design, manufacturing and end user costs, operability and/or patient use.

The use of volatile liquids as active agents or comprising active agents is known. One such example is halogenated volatile liquids. Halogenated volatile liquids have been described as useful for inducing and/or maintaining anaesthesia (including amnesia, muscle paralysis, and/or sedation) and/or analgesia and may therefore be useful as anaesthetics and/or analgesics. The anaesthetic properties of fluorinated compounds have been known since at least 1946 (Robbins, B. H. J Pharmacol Exp Ther (1946) 86: 197-204). This was followed by the introduction of fluoroxene, halothane and methoxyflurane into clinical use in the 1950s and the subsequent development of enflurane, isoflurane, sevoflurane and desflurane which are in clinical use in some countries today (Terrell, R. C. Anesthesiology (2008) 108 (3): 531-3).

Halogenated volatile liquids, when used for general anaesthesia, may be delivered to a patient under positive pressure via a delivery system that includes a vaporizer and a flow of breathable carrier gas. More recently, halogenated volatile liquids have been formulated for use in local or regional anaesthesia and delivery via non-inhalation routes. Examples include formulation as: microdroplets for intradermal or intravenous injection (e.g. U.S. Pat. No. 4,725,442); aqueous solutions for intrathecal or epidural delivery (e.g. WO2008/036858); swab, droplets, spray or aerosol for transmucosal delivery (e.g. WO2010/025505); aqueous based solutions comprising an extractive solvent in an amount effective to reduce the volatility, vaporisation or evaporation of the volatile anaesthetic for transdermal, topical, mucosal, buccal, rectal, vaginal, intramuscular, subcutaneous, perineural infiltration, intrathecal or epidural delivery (e.g. WO2009/094460, WO2009/094459); compositions suitable for formulation into a medical patch (e.g. WO2014/143964); compositions suitable for formulation as a solution, suspension, cream, paste, oil, lotion, gel, foam, hydrogel, ointment, liposome, emulsion, liquid crystal emulsion and nanoemulsions for topical, intrathecal, epidural, transdermal, topical, oral, intra-articular, mucosal, buccal, rectal, vaginal, intramuscular, intravesical and subcutaneous delivery (e.g. WO2008/070490, WO2009/094460, WO2010/129686); and stable and injectable liquid formulations (WO2013/016511).

The main consideration(s) for the safe storage and handling of volatile liquids commonly include vapour pressure build up, the robustness of the container and the integrity of the container seal(s). The chemical nature of the volatile liquid may also be important if the active agent is capable of permeating, solubilizing or otherwise reacting with the container material(s) upon storage. A number of storage containers for halogenated volatile liquids have been described including: rigid polymeric containers as a replacement for glass vials, such as capped bottles large tanks, shipping containers (e.g. WO1999/034762, WO2012/116187); rigid polymeric bottles fitted with a gasketless valve assembly and pliable containers with a threaded spout for fluid connection to deliver liquid anaesthetics to an anaesthetic machine or vaporizer (e.g. WO2010/135436, WO2013/106608, WO2013/149263, WO2015/034978); a container with a capped membrane for delivering a stored liquid anaesthetic to a vaporizer via a slotted tube (WO2009/117529); and rigid polymeric and aluminium containers optionally coated with materials to impart or enhance vapour barrier characteristics or container inertness (e.g. WO2002/022195, WO2003/032890, WO2010/129796).

Despite the various advances in formulating volatile liquids in non-inhalable forms, such as the halogenated volatile liquids, as well as containers to store them, there still remains a need for inhalable forms of volatile liquids and devices to store and/or administer them to patients.

Attempts to design new inhalers for inhalable medicines in general are ongoing. For example, WO2008/040062 describes a diverse number of inhaler device concepts that depend on complex constructions and moving parts for storing and/or delivering inhalable liquids and powdered solids into a user's mouth or nose. The various devices described are adapted to hold one or two medicament containers in the form of pressurised canisters, ampoules, vials and plungers. The devices are described as being activated by sliding an outer wall of the device in relation to an inner wall of the device to deliver the liquid medication from a medication container. In a number of embodiments, the device includes a moveable mouthpiece which deploys in order to open the air pathway. The device is also described as including one or more one-way valves to provide a unidirectional air flow for one or both inhaled air and exhaled air (a series of one-way valves to direct the flow of inhaled and exhaled air has also been generally described in WO2007/033400 which is an incorporation by reference of the device described in WO1997/003711).

When required for use, the devices of WO2008/040062 are claimed as being capable of releasing the medication by punching means namely two punches to perforate the two frangible ends respectively of a medication container having frangible ends, although various other means are generally described including: pressurised means (e.g. by a pressurised canister); frangible means (e.g. by rupturing an ampoule with a striker or by punching a frangible membrane or seal of a vial with punch means); crushable means (e.g. by crushing a vial with a plunger); dislodging means (e.g. by dislodging an unscrewed cap from a vial); and plunging means (e.g. by plunging the medication from the plunger barrel).

However, inhalable liquids such as halogenated volatile liquids require an effective air chamber into which the vapour may evaporate and allow an effective airflow through the air/vapour chamber for delivery to a patient. Accordingly, embodiments such as those described in, for example, FIGS. 48A, 48B, 48C, 49A, 49B, 50A, 50B, 51A, 51B, 56A, 56B, 57, 58A, 58B, 58C and 58D of WO2008/040062, would not be expected to work in practice as the evaporative means (or wick) is prevented from being effectively exposed to the released liquid by the walls of the liquid storage container itself.

The present invention provides a new inhaler device for the storage and administration of inhalable liquids to a patient offering one or more advantages or improvements over known inhalers, particularly inhalers for the delivery of halogenated volatile liquids such as methoxyflurane for use as an analgesic. The device is capable of storing and administering an inhalable liquid with a minimum of three manufactured parts (excluding the passive evaporation support material pre-loaded with the inhalable liquid). The device offers an easy to use, pre-loaded (i.e. primed for use), readily portable and low-cost manufactured device which may also provide further reductions in shipping, storage and disposal costs as well as material wastage, by avoiding the need to store the liquid in a separately manufactured container.

### SUMMARY

According to a first aspect of the invention there is provided an inhaler device for the storage and delivery of an inhalable liquid to a patient, said device comprising:
(1) A sealed elongated body having a first end and a second end wherein the sealed elongated body is re-sealable, partially re-sealable or non-resealable;
(2) A first end-seal for sealing the first end of the elongated body;
(3) A second end-seal for sealing the second end of the elongated body; and
(4) A passive evaporation support material pre-loaded with the inhalable liquid;

wherein the first end-seal and the second end-seal are independently selected from a resealable end-seal or a non-resealable end-seal and further wherein as the inhalable liquid forms a vapour upon storage, the elongated body forms a vapour chamber such that the stored vapour is available for direct administration to a patient upon opening the first and second end-seals.

According to a second aspect of the invention there is provided an inhaler device for the storage and delivery of an inhalable liquid to a patient, said device consisting only of:
(1) A sealed elongated body having a first end and a second end wherein the sealed elongated body is re-sealable, partially re-sealable or non-resealable;
(2) A first end-seal for sealing the first end of the elongated body;
(3) A second end-seal for sealing the second end of the elongated body; and
(4) A passive evaporation support material pre-loaded with the inhalable liquid;

wherein the first end-seal and the second end-seal are independently selected from a resealable end-seal or a non-resealable end-seal and further wherein as the inhalable liquid forms a vapour upon storage, the elongated body forms a vapour chamber such that the stored vapour is available for direct administration to a patient upon opening the first and second end-seals.

In one embodiment according to the first and second aspects of the invention, the sealed elongated body is resealable and the first and second end-seals are both resealable. In an alternative embodiment the sealed elongated body is non-resealable and the first and second end-seals are non-resealable. In yet another embodiment the sealed elongated body is partially resealable and the first end-seal is a resealable end and the second end-seal is a non-resealable end and vice versa.

In one embodiment according to the first and the second aspects, the inhalable liquid is a halogenated volatile liquid. In a further embodiment the halogenated volatile liquid is selected from the group consisting of halothane (2-bromo-2-chloro-1,1,1-trifluoroethane), sevoflurane (fluoromethyl-2,2,2-trifluoro-1-(trifluroromethyl)ethyl ether), desflurane (2-difluoromethyl-1,2,2,2-tetrafluoroethrylether), isoflurane (1-chloro-2,2,2-trifluoroethyldifluoromethyl ether), enflurane (2-chloro-1,1,2-trifluoroethyldifluoromethyl ether) and methoxyflurane (2,2-dichloro-1,1-difluoroethylmethyl ether). In a preferred embodiment, the inhalable liquid is methoxyflurane for use as an analgesic.

### BRIEF DESCRIPTION OF THE FIGURES

FIG. 1 shows a prior art inhaler device, referred to as the Green Whistle™ inhaler device (Medical Developments International Limited) that is currently used to administer methoxyflurane.

FIG. 2 shows two examples of inhaler devices according to embodiments of the invention comprising a first resealable sealed end and a second resealable sealed end (FIGS. 2A & 2B).

FIG. 3 shows the inhaler device of FIG. 2A when viewed from above and showing the direction of air intake into the air inlet hole(s) in the resealable second sealed (air inlet) end in the form of an adjustable end cap in an open or ‘activated’ position (FIG. 3A). FIG. 3B is a cross-sectional view of the device shown in FIG. 3A with the adjustable end cap in a closed or ‘sealed’ position. FIG. 3C shows an enlarged view of the adjustable end cap of FIG. 3B.

FIG. 4 shows an exploded view of the inhaler device of FIG. 2B (FIG. 4A) together with a cross-sectional view of the device in storage mode (FIG. 4B) and administration mode (FIG. 4C).

FIG. 5 shows the elongated body of the inhaler device of FIG. 2B to illustrate the external screw thread arrangement (FIG. 5A) adapted to matingly engage with an adjustable mouthpiece end cap as shown when viewed from the top (FIG. 5B) and line drawing views to illustrate the internal screw thread arrangements (FIGS. 5C and 5D) including cross-sectional view A-A of the adjustable mouthpiece end cap in FIG. 5D (FIG. 5E).

FIG. 6 shows the elongated body of the inhaler device of FIG. 2A or FIG. 2B to illustrate the external screw thread arrangement (FIG. 6A) adapted to matingly engage with an adjustable air inlet end cap having two air inlet holes (FIG. 6B) or four inlet holes (FIG. 6C).

FIG. 7 shows an inhaler device according to an embodiment of the invention comprising a first non-resealable sealed end and a second non-resealable sealed end (FIG. 7A) wherein the first and second non-resealable sealed ends are the same. An enlarged view (FIG. 7B) and a perspective view (FIG. 7C) of the non-resealable sealed ends is presented.

FIG. 8 shows an inhaler device according to an embodiment of the invention comprising a first non-resealable sealed end and a second non-resealable sealed end in administration mode (FIGS. 8A and 8B).

FIG. 9 provides further illustrations of the device of FIG. 8 and its components including a perspective view of the elongated body (FIG. 9A), a perspective view of the assembled device (FIG. 9B) and cross-sectional views A-A of the device in FIG. 9B (FIGS. 9C and 9D).

FIG. 10 shows a perspective view (FIG. 10A) and a top view (FIG. 10B) of an example of passive evaporation support material according to an embodiment of the invention that comprises three or more longitudinal conduits wherein the conduits are formed by the passive evaporation support material together with an internal surface of the elongated body.

### DETAILED DESCRIPTION

Inhaler devices that are useful for administering inhalable liquids may be generally considered to operate by either passive or active means in order to deliver the active agent(s) to a patient. Inhaler devices with active means may include pressurized, moving, mechanical, heating and/or electrical means to, for example, nebulise, vaporize and/or generally deliver the active agent(s). In contrast, inhaler devices with passive means rely solely on the vaporisation or evaporation of the active agent(s) at ambient conditions and respiration of the patient to deliver the active agent(s).

The Analgizer™ inhaler device (Abbott Laboratories Corporation) is an example of a device that operates by passive means to deliver an inhalable liquid. According to the USPTO TESS database, the Analgizer™ was a registered, now lapsed, trademark in respect of an inhaler for the supervised self-administration of inhalation anaesthesia and was first used in 1968. The Analgizer™ was a very simple device that consisted of a white cylindrical polyethylene open-ended tube having a mouthpiece and an absorbent wick of polypropylene which was tightly rolled into a ‘Swiss-roll’ shape, i.e. cross-sectional view. The inhalation anaesthetic, methoxyflurane (15 mL), was poured into the open ended base of the inhaler and onto the tightly wound wick, just prior to use. A patient was then able to self-administer the liquid anaesthetic by inhaling through the mouthpiece.

The Green Whistle™ inhaler device (Medical Developments International Limited) was subsequently developed during the 1990s and has since been used in Australia for the delivery of Penthrox®/™ (methoxyflurane) as an analgesic (1.5 mL or 3 mL, storage brown glass vial container with screw cap). Although similar in its simplicity of design to the Analgizer™, the Green Whistle™ device includes certain functional improvements such as the inclusion of a one-way valve at the base end to prevent drug vapour loss from the device upon patient exhalation and an activated carbon (‘AC’) chamber designed to be externally fit into a dilution hole in the mouth piece to filter exhaled drug vapours. Additional design modifications to the base end included the introduction of cap lugs to assist removal of the cap from the glass vial used to store the drug dose to be delivered, a dome to facilitate the spread of the poured liquid onto the ‘S-shaped’ wick (i.e. cross-sectional view) or, in the alternative to a dome, an inlet nipple to allow for the attachment of a breathable gas line to direct the gas through the device. The Green Whistle™ device is designed for single patient use.

Methoxyflurane (Penthrox®/™, Medical Developments International Limited) offers a non-narcotic, i.e. non-opioid analgesic alternative to common analgesics such as morphine and fentanyl. Methoxyflurane also presents an alternative to analgesics which are administered in oral tablet form or intravenously to a patient and may therefore be particularly useful when rapid pain relief is required in clinical, surgical (e.g. pre- and post-operative) and/or emergency settings (e.g. emergency department and triage management as well as by first-responders such as paramedics and search and rescue teams). However, the Green Whistle™ device is currently the only device that is commercially available to administer methoxyflurane. According to the device's instructions for use, the administrator is required to hold the methoxyflurane bottle upright to use the base of the inhaler to loosen the bottle cap and then to remove the cap by hand before tilting the inhaler to a 45° angle and pouring the contents of the bottle into the base while rotating the device. An AC-chamber may be optionally fitted externally to the device either beforehand or afterwards. While the device is effective, the number of steps and separate components may present handling difficulties for the administrator or self-administrator, for example, in high-stress and/or emergency settings.

The present invention provides a new inhaler device for the storage and administration of inhalable liquids to a patient, such as halogenated volatile liquids, particularly methoxyflurane for use as an analgesic, the device having one or more advantages or improvements over known inhalers.

### Definitions

Unless otherwise herein defined, the following terms will be understood to have the general meanings which follow.

‘Active agent’ refers to therapeutic agents and non-therapeutic agents and compounds, formulations and compositions comprising them.

‘Alleviate’, ‘Alleviation’ and variations thereof refers to relieving, lessening, reducing, ameliorating or an improvement in the symptom(s) and/or underlying cause(s) of a condition and/or disease in a patient.

‘Delivery dose’ refers to the dose of inhalable liquid or active agent for administration to a patient.

‘Filter’, ‘Filtering’ and variations thereof refers to the ability of a substance to absorb, adsorb, capture, trap, scavenge, scrub or partially or entirely remove the inhalable volatile liquid vapour from the exhaled breath of a patient upon exhalation.

‘Halogenated volatile liquids’ refers to volatile liquids which (i) comprise at least one halogen atom selected from the group consisting of a chlorine (CI), bromine (Br), fluorine (F) and iodine (I) atoms, or (ii) comprise an active agent which comprises at least one halogen atom selected from the group consisting of a chlorine (CI), bromine (Br), fluorine (F) and iodine (I) atoms. In some embodiments, halogenated, particularly fluorinated, hydrocarbons and halogenated, particularly fluorinated, ethers may be preferred. In some embodiments, halogenated ethers may be particularly preferred and include but are not limited to, halothane (2-bromo-2-chloro-1,1,1-trifluoroethane), sevoflurane (fluoromethyl-2,2,2-trifluoro-1-(trifluroromethyl)ethyl ether), desflurane (2-difluoromethyl-1,2,2,2-tetrafluoroethrylether), isoflurane (1-chloro-2,2,2-trifluoroethyldifluoromethyl ether), enflurane (2-chloro-1,1,2-trifluoroethyldifluoromethyl ether) and methoxyflurane (2,2-dichloro-1,1-difluoroethylmethyl ether).

‘Inhalable liquid’ refers to liquids that comprise active agents or that are themselves the active agent and that are readily inhalable or capable of being or adapted to be inhaled by a patient. In some embodiments, inhalable volatile liquids, particularly halogenated volatile liquids are preferred.

‘Inhalation’, ‘Inhalable’ and variations thereof refers to the intake of, for example but not limited to air, breathable gases, inhalable liquids, by a patient and includes both oral and nasal inhalation. In some embodiments, oral inhalation is particularly preferred.

‘Patient’ refers to both human and veterinary patients. In some embodiments, human patients may be particularly preferred. Reference to a patient will therefore be understood to mean the person or animal to whom the inhalable liquid is administered to and in the case of human patients, will be understood to include administration by self-administration.

‘Pharmaceutical agent’ refers to a drug, or a compound, formulation or composition that comprises a drug, for the treatment of symptom(s) and/or underlying cause(s) of a condition and/or disease in a patient. The term pharmaceutical agent may be used interchangeably with therapeutic agent or active agent.

‘Respiratory’, ‘Respirational’ and variations thereof refers to the act of respiring, breathing, inhaling and exhaling, such as for example but not limited to air, breathable gases, inhalable liquids and active ingredients, by a patient.

‘Room temperature’ refers to ambient temperatures which may be, for example, between 10° C. to 40° C. but more typically between 15° C. to 30° C.

‘Therapeutic agent’ refers to an active agent, or a compound, formulation or composition (including biological compounds, formulations and compositions) that comprises an active agent, that is capable of treating a patient or offers a therapeutic or medical benefit to a patient or that has or that requires regulatory and/or marketing approval for therapeutic use in a patient. Therapeutic agents include pharmaceutical agents. In contrast, a ‘Non-therapeutic agent’ will be understood to mean an active agent which may not have or require regulatory and/or marketing approval for a therapeutic use such as, for example, smokeless tobacco products and electronic cigarettes, or does not have a recognised or identified therapeutic use but may be used by a patient for a non-therapeutic reason such as general health, wellbeing or physiological benefit such as, for example, nutraceutical products.

‘Treat’, ‘Treatment’ and variations thereof refers to the alleviation, modulation, regulation or halting of the symptom(s) and/or underlying cause(s) of a condition and/or disease in a patient. In some embodiments treatment may include preventative or prophylactic treatment.

‘Volatile liquids’ refers to substances that predominantly exist in a liquid form but readily form vapours, evaporate or vaporize such that they partially exist in a vapour form under ambient conditions for example, at room temperature and at normal atmospheric pressures.

### EMBODIMENTS

Embodiments will now be described with reference to the non-limiting examples.

There is provided an inhaler device for the storage and delivery of an inhalable liquid to a patient, said device comprising:
(1) A sealed elongated body having a first end and a second end wherein the sealed elongated body is resealable, partially resealable or non-resealable;
(2) A first end-seal for sealing the first end of the elongated body;
(3) A second end-seal for sealing the second end of the elongated body; and
(4) A passive evaporation support material pre-loaded with the inhalable liquid;

wherein the first end-seal and the second end-seal are independently selected from a resealable end or a non-resealable end and further wherein as the inhalable liquid forms a vapour upon storage, the elongated body forms a vapour chamber such that the stored vapour is available for direct administration to a patient upon opening the first and second end seals.

In another embodiment there is provided an inhaler device for the storage and delivery of an inhalable liquid to a patient, said device consisting only of:
(1) A sealed elongated body having a first end and a second end wherein the sealed elongated body is re-sealable, partially re-sealable or non-resealable;
(2) A first end-seal for sealing the first end of the elongated body;
(3) A second end-seal for sealing the second end of the elongated body; and
(4) A passive evaporation support material pre-loaded with the inhalable liquid;

wherein the first end-seal and the second end-seal are independently selected from a resealable end or a non-resealable end and further wherein as the inhalable liquid forms a vapour upon storage, the elongated body forms a vapour chamber such that the stored vapour is available for direct administration to a patient upon opening the first and second end-seals.

In one embodiment the sealed elongated body is resealable and the first and second end-seals are both resealable. In storage mode the resealable end-seals are closed. When required for use, the resealable end-seals are opened to provide an air flow pathway through the device and deliver the vapour from the vapour chamber to the user when the user inhales.

In one embodiment the first end-seal and the second end-seal are resealable end-seals independently selected from a plug, an end cap or adjustable end cap comprising at least one air inlet opening. The end cap and the adjustable end cap may be detachably fastened to rotatingly engage with the rest of the elongated body of the device by, for example, a screw thread arrangement or a snap-fit joint arrangement. The plug may be detachably fastened in the same way or by virtue of a tight tolerance fit with the elongated body.

When the resealable end-seal is an adjustable end cap, the air inlet opening(s) may be formed in the adjustable end cap in a number of ways when the adjustable end cap is opened, for example, by groove(s) or hole(s) which may be exposed to provide an air flow pathway or by groove(s) or hole(s) which may optionally align with groove(s) or hole(s) in the elongated body. Accordingly, in one embodiment the resealable end-seal is an adjustable end cap comprising at least one air inlet opening independently selected from a groove or a hole.

When the device is required for patient use, the adjustable end cap may be gradually adjusted from a closed position where it completely covers the air inlet opening(s), to a partially opened or fully opened position to enable the air to flow into the vapour chamber and across the surface(s) of the passive evaporation support material to deliver the vapour to the patient as the patient inhales. In use, the air inlet opening(s) may be opened by opening the adjustable end cap in a number of ways, for example, by popping, upward pulling, twisting, turning, rotating or unscrewing the adjustable end cap relative to the elongated body. The air flow pathway may be adjustably controlled by the degree of popping, upward pulling, twisting, turning, rotating or unscrewing of the adjustable end cap relative to the elongated body to provide partially opened or fully opened air inlet opening(s).

The resealable end-seal may optionally comprise a wad insert to assist with sealing and resealing the device for storage mode. The wad insert may comprise a compressible material and a vapour impermeable film or foil to assist with providing a tight seal when the resealable end is closed. Examples of compressible materials include but are not limited to polymeric foams or sponges such as LDPE. Examples of vapour impermeable films include but are not limited to polymeric films such as PET and metal foils such as aluminium, nickel and alloys thereof. In one embodiment the end cap optionally comprises a wad insert.

In another embodiment the sealed elongated body is non-resealable and the first end-seal and second end-seal are non-resealable end-seals. When required for use, the non-resealable end-seals are irreversibly opened to provide an air flow pathway through the device and deliver the vapour from the vapour chamber to the user when the user inhales. Examples of non-resealable end-seals may include but are not limited to crown seals (including ring-pull crown caps) and a vapour impermeable film or foil. The non-resealable end-seals may be opened, for example, by pulling, tearing, ripping, peeling, perforating, puncturing or piercing. The non-resealable end-seals may therefore optionally comprise a pulling, tearing, ripping, peeling, perforating, puncturing or piercing means to open the seal. In one embodiment the first end-seal and second end-seal are non-resealable end-seals independently selected from the group consisting of crown seals, a vapour impermeable film or foil. In one embodiment the non-resealable end-seal is a crown seal, preferably a ring-pull crown cap. In another embodiment the non-resealable end-seal is a vapour impermeable film or foil.

Examples of vapour impermeable films include but are not limited to polymeric films, metal foils (such as, for example, aluminium, nickel and alloys thereof) and combinations, including co-extruded polymeric films and/or foils such as laminate films, thereof. In one embodiment the vapour impermeable film is a single layer selected from a polymeric film or a metal foil. In another embodiment the vapour impermeable film is a laminate film comprising two or more layers selected from a polymeric film, a metal foil and combinations, including co-extruded polymeric films and/or foils, thereof. The laminate film may comprise a weldable layer made from a suitable weldable foil or polymeric film such as, for example, LLDPE. A weldable layer may assist with sealing the layers of a laminate together and/or sealing a vapour impermeable film comprising a weldable layer to the device. Processes suitable for welding include thermal and ultrasonic welding.

In one embodiment the polymeric film has a MVTR of less than 100 g/m²/24 h, preferably less than 50 g/m²/24 h. In one embodiment the polymeric film comprises a polymer selected from the group consisting of a polyolefin, a polymeric phthalate, a fluorinated polymer, a polyester, a nylon, a polyvinyl, a polysulfone, a natural polymer and combinations, including co-extruded polymers thereof including biaxially orientated polymers such as, for example, biaxially orientated polypropylene (BOPP). In one embodiment the polymeric film comprises a polymer selected from the group consisting of PP, PE, LDPE, LLDPE, HDPE, BOPP, 4-methylpentene, polymethylpentene polycyclomethylpentene, PEN, PET, PETP, PEI, PBT, PTT, PCT, Kel-F, PTFE, cellulose acetate, POM, PETG, PCTG, PCTA, nylon, PVA, EVOH, starch, cellulose, proteins and combinations, including co-extruded polymers, thereof.

In one embodiment the vapour impermeable film comprises PET. In another embodiment the vapour impermeable film comprises PET and a metal foil layer, preferably an aluminium foil layer. In one embodiment the vapour impermeable film comprises metalised PET (Met PET).

In yet another embodiment sealed elongated body is partially-resealable and the first end-seal is a resealable end-seal and the second sealed end is a non-resealable end-seal and vice versa. When required for use, the resealable end-seal is opened and the non-resealable end-seal is removed to provide an air flow pathway through the device and deliver the vapour from the vapour chamber to the user when the user inhales.

The first end-seal and second end-seal may be the same or different. When the first and second end-seals are the same, the device may be adapted for orientation in either direction for use. However, one sealed end may be specifically adapted to function as an air inlet end comprising at least one air inlet opening and the other sealed end may be specifically adapted to function as a mouthpiece end comprising at least one vapour inhalation opening, in which case the air inlet end and the device may require a specific orientation for use. For example, one sealed end may be specifically adapted to function as a mouthpiece end and comprise at least a portion that tapers to a vapour inhalation opening.

Accordingly, it will be understood that when the device is in use, one end will function as an air inlet end comprising at least one air inlet opening and the other end will function as a mouthpiece end comprising at least one vapour inhalation opening. The elongated body may optionally comprise one or more openings, for example, groove(s) or hole(s), adapted to partially or fully align with the air inlet opening(s) in the air inlet end when in use. When an end of the elongated body is sealed by a resealable end-seal, the air inlet opening(s) and/or vapour inhalation opening(s) may be independently formed in the end-seals(s) to provide an air flow pathway through the device when the resealable end-seal(s) is/are opened or when the opening(s) is/are partially or fully aligned with opening(s) in the elongated body to deliver the vapour from the vapour chamber to the user when the user inhales. When an end of the elongated body is sealed by a non-resealable end-seal, the air inlet opening(s) and/or vapour inhalation opening(s) may be independently formed by the circumference of the ends of the elongated body to provide an air flow pathway through the device when the non-resealable end-seal(s) is/are opened or removed deliver the vapour from the vapour chamber to the user when the user inhales.

The present device comprises a passive evaporation support material pre-loaded with the inhalable liquid to provide a portable, ready-to-use, all-in-one, drug storage and delivery device. In comparison to the prior inhaler devices for methoxyflurane, the present device provides easy administration, in particular self-administration when rapid pain relief is required, for example, in emergency, non-hospital, isolated, outdoor environment, sporting, humanitarian aid and/or field operation environments.

In one embodiment the passive evaporation support material is adapted to form a single longitudinal airflow/vapour pathway though the vapour chamber. In another embodiment, the passive evaporation support material is adapted to form at least two independent longitudinal airflow/vapour pathways though the vapour chamber. In yet another embodiment, the passive evaporation support material is adapted to form three or more independent longitudinal airflow/vapour pathways though the vapour chamber.

In one embodiment the passive evaporation support material is adapted to form a single longitudinal airflow/vapour pathway though the vapour chamber, the form being selected from the group consisting of a planar lining; a partial lining of the vapour chamber walls; and a full lining of the vapour chamber walls.

In another embodiment the passive evaporation support material is adapted to form at least two independent longitudinal airflow/vapour pathways, preferably three or more independent longitudinal airflow/vapour pathways, through the vapour chamber. Numerous examples of cross-sectional shapes which are capable of forming at least two, preferably three or more independent longitudinal airflow/vapour pathways may be envisaged, some of which follow.

The two, three or more independent longitudinal airflow/vapour pathways may be formed by the passive evaporation support material adopting a cross-sectional shape selected from a letter of the alphabet or a single digit number such as, for example although not limited to, an A-shape, B-shape, S-shape, Z-shape, figure-2, figure-5 and figure-8 which are capable of forming at least two independent airflow/vapour pathways, and a K-shape, M-shape, V-shape, W-shape, X-shape, Y-shape and figure-3 which are capable of forming three or more independent longitudinal airflow/vapour pathways through the vapour chamber.

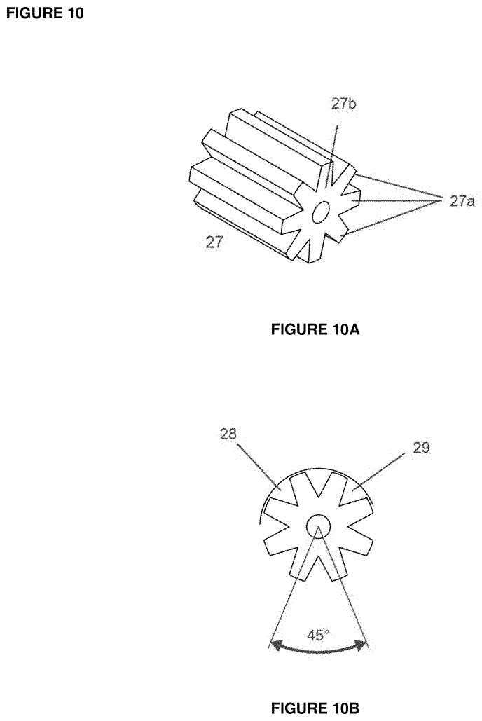

In one embodiment the passive evaporation support material is adapted to provide three or more independent longitudinal airflow/vapour pathways. The pathways may be formed as independent conduits through the passive evaporation support material itself or the pathways may be formed by the evaporative means making contact with an internal surface of the vapour chamber. Accordingly, in one embodiment, the passive evaporation support material comprises three or more longitudinal conduits wherein the conduits are formed within the passive evaporation support material or are formed by the passive evaporation support material together with an internal surface of the vapour chamber or a combination thereof. One example is shown in FIG. 10 whereby the passive evaporation support material (27) comprises three or more radial arms (27a) extending from a central portion (27b) to an internal surface of the vapour chamber (28) to form three or more longitudinal conduits (29). Passive evaporation support material which are adapted to provide three or more independent longitudinal airflow/vapour pathways may be particularly suited to smaller sized devices.

The passive evaporation support material may be made from any material that is suitable for absorbing the inhalable liquid and passively releasing it as a vapour. Materials which have wicking properties may be particularly suitable passive evaporation support materials for use in the present device. Wicking properties will generally be understood to include the ability of a material to facilitate or enhance the rate of evaporation or vaporisation of a liquid from its surface by distributing the liquid, whether by drawing, spreading, pulling or otherwise, throughout the material from its initial point of contact and/or as it evaporates from an exposed surface area of the material. Accordingly, in one embodiment the passive evaporation support material is a wicking material. In one embodiment the wicking material is a wicking felt or a porous polymeric material. In a preferred embodiment the wicking material is a polypropylene wicking felt.

The present device is considered to be particularly useful for storing and administering a halogenated volatile liquid, particularly methoxyflurane for use as an analgesic. Accordingly, in one embodiment the inhalable liquid is a halogenated volatile liquid. In a further embodiment the halogenated volatile liquid is selected from the group consisting of halothane (2-bromo-2-chloro-1,1,1-trifluoroethane), sevoflurane (fluoromethyl-2,2,2-trifluoro-1-(trifluroromethyl)ethyl ether), desflurane (2-difluoromethyl-1,2,2,2-tetrafluoroethrylether), isoflurane (1-chloro-2,2,2-trifluoroethyldifluoromethyl ether), enflurane (2-chloro-1,1,2-trifluoroethyldifluoromethyl ether) and methoxyflurane (2,2-dichloro-1,1-difluoroethylmethyl ether). In a preferred embodiment, the inhalable liquid is methoxyflurane for use as an analgesic.

Suitable delivery doses of inhalable liquid for administration to a patient by the present device may be determined by reference to, for example, regulatory approved dosage amounts. Suitable delivery doses of methoxyflurane for use as an analgesic will typically be less than 15 mL and preferably less than 12 mL. In one embodiment the delivery dose is selected from the group consisting of 0.5 mL, 1 mL, 1.5 mL, 2 mL, 2.5 mL, 3 mL, 3.5 mL, 4 mL, 4.5 mL, 5 mL, 5.5 mL, 6 mL, 6.5 mL, 7 mL, 7.5 mL, 8 mL, 8.5 mL, 9 mL, 9.5 mL, 10 mL, 10.5 mL, 11 mL, 11.5 mL and 12 mL. In one embodiment the delivery dose of methoxyflurane for administration by the present device is selected from the group consisting of 1.5 mL, 3 mL and 6 mL.

The device may be made from various materials. However, suitable material(s) may be selected by considering whether they are chemically inert, stable and impervious with reference to the inhalable liquid to be stored and/or delivered. Material(s) may also be selected based on their suitability for medical device applications such as by reference to whether they meet approved standards for medical-grade human use by a regulatory authority like the FDA.

It is envisaged that the present device will be particularly useful for storing and administering halogenated volatile liquids. Accordingly, in one embodiment, the device is made from one or more materials that are compatible with the storage and delivery of halogenated volatile liquids to a patient, in particular methoxyflurane for use as an analgesic.

Examples of materials which may be suitable for making the present device include but are not limited to polymers (including homopolymers and heteropolymers i.e. co-polymers), composites (including nanocomposites), metals (including alloys thereof) and combinations thereof. In one embodiment, the device is made from polymers (including homopolymers and heteropolymers i.e. co-polymers), composites (including nanocomposites such as polymers in combination with clay), metals (including aluminium and alloys thereof) and combinations thereof. In a further embodiment, the device is optionally internally lined or coated with one or more material(s) selected from the group consisting polymers (including homopolymers and heteropolymers i.e. co-polymers), composites (including nanocomposites such as polymers in combination with clay), metals (including aluminium, nickel and alloys thereof), oxides (including aluminium oxides, silicon oxides), resins (including epoxyphenolic resins and ionomeric resins such as Surlyn®, trademark of DuPont), lacquers and enamels.

It is considered that one advantage of the present device is its relative simplicity and low cost to manufacture in addition to ease of operability in terms of the minimum number of individual components or parts required for the storage and administration of the inhalable liquid. The elongated body of the device may be formed as a single manufactured part. The end-seals may be separately formed from the same or a different material. In one embodiment the elongated body, the first end-seal and the second end-seal are independently made from a material selected from the group consisting of a polymeric material, a metal (for example, aluminium, nickel) and a metal alloy (for example, stainless steel).

Polymers are particularly suited to large scale manufacturing of the present device and polymeric films described herein by injection moulding, blow moulding and extrusion processes. They may also be suitable for manufacturing the present device on a smaller scale by 3D printing techniques. Further, polymers may be recycled following disposal of the device.

Examples of polymers for use in making the present device and polymeric films described herein may include but are not limited to the following polymers and combinations (including co-extruded polymers) thereof: polyolefins such as polypropylene (‘PP’), polyethylene (‘PE’) including low density (‘LDPE’), linear low density (‘LLDPE’) and high density polyethylene (‘HDPE’), biaxially orientated polypropylene (‘BOPP’), 4-methylpentene, polymethylpentene, polycyclomethylpentene; polymeric phthalates such as polyethylene naphthalates (‘PEN’), polyethylene terephthalate (‘PET’) (also known as (‘PETE’)), polyethylene terephthalate polyester (‘PETP’), polyethylene isophthalate (‘PEI’), polybutylene terephthalate (‘PBT’), polytrimethylene terephthalate (‘PTT’), polycyclohexylenedimethylene terephthalate (‘PCT’); fluorinated polymers including polymers fluorinated after manufacture (e.g. fluorination post-moulding), fluorinated ethylene-propylene, chlorotrifluoroethylene (‘Kel-F’), polytetrafluoroethylene (‘PTFE’); polyesters including cellulose acetate, polyoxymethylene (‘POM’) and polyesters containing a terephthalate ester group including co-polymers such polyethylene terephthalate glycol co-polyester (‘PETG’), polycyclohexylenedimethylene terephthalate glycol modified (‘PCTG’) and polycyclohexylenedimethylene terephthalate/isophthalic acid (‘PCTA’); nylons including amorphous nylon; polyvinyls including polyvinyl alcohol (‘PVA’) and ethylene vinyl alcohol (‘EVOH’); polysulfones including polyethersulfone (‘PES’); and natural polymers including starch, cellulose and proteins. Suitable polymers may also include polymers with a moisture vapour transmission rate (‘MVTR’, also known as water vapour transmission rate ‘WVTR’) of less than 100 g/m²/24 h, preferably less than 50 g/m²/24 h.

Accordingly, in one embodiment the device is made from one or more polymers wherein the device further comprises an optional internal lining or coating with one or more material(s) selected from the group consisting polymers (including homopolymers and heteropolymers (also known as co-polymers) and combinations thereof including co-extruded polymers), composites (including nanocomposites such as polymers in combination with clay), metals (including aluminium, nickel and alloys thereof), oxides (including aluminium oxides, silicon oxides), spray coatings, resins (including epoxyphenolic resins and ionomeric resins such as Surlyn®, trademark of DuPont), lacquers and enamels.

In one embodiment the polymer is selected from a polyolefin, a polymeric phthalate, a fluorinated polymer, a polyester, a nylon, a polyvinyl, a polysulfone, a natural polymer and combinations, including co-extruded polymers thereof. In one embodiment the polymer has a MVTR of less than 100 g/m²/24 h, preferably less than 50 g/m²/24 h. In one embodiment the polyolefin is selected from the group consisting of PP, PE, LDPE, LLDPE, HDPE, 4-methylpentene, polymethylpentene polycyclomethylpentene and combinations, including co-extruded polymers thereof such as BOPP. In one embodiment the polymeric phthalate is selected from the group consisting of PEN, PET, PETP, PEI, PBT, PTT, PCT and combinations, including co-extruded polymers, thereof. In one embodiment the fluorinated polymer is selected from Kel-F, PTFE and combinations, including co-extruded polymers thereof. In one embodiment the polyester is selected from the group consisting of cellulose acetate, POM and polyesters containing a terephthalate ester group including PETG, PCTG, PCTA and combinations, including co-extruded polymers, thereof. In one embodiment the nylon is an amorphous nylon. In one embodiment the polyvinyl is selected from PVA, EVOH and combinations, including co-extruded polymers, thereof. In one embodiment the polysulfone is PES. In one embodiment the natural polymer is selected from the group consisting of starch, cellulose, proteins and combinations, including co-extruded polymers, thereof.

In one embodiment the device is made from a single polymer selected from the group consisting of PP, PE, LDPE, LLDPE, HDPE, BOPP, 4-methylpentene, polymethylpentene polycyclomethylpentene, PEN, PET, PETP, PEI, PBT, PTT, PCT, Kel-F, PTFE, cellulose acetate, POM, PETG, PCTG, PCTA, nylon, PVA, EVOH, starch, cellulose, proteins and combinations, including co-extruded polymers, thereof. In another embodiment the device is made from two or more polymers selected from the group consisting of PP, PE, LDPE, LLDPE, HDPE, 4-methylpentene, polymethylpentene polycyclomethylpentene, PEN, PET, PETP, PEI, PBT, PTT, PCT, Kel-F, PTFE, cellulose acetate, POM, PETG, PCTG, PCTA, nylon, PVA, EVOH, starch, cellulose, proteins and combinations, including co-extruded polymers, thereof. In one embodiment, the device is made from a polymer selected from the group consisting of HDPE, PET and combinations thereof. In one embodiment the device comprises PET.

The elongated body of the device may generally adopt the same cross-sectional shape along its length. In one embodiment the cross-sectional shape of the elongated body is selected from the group consisting of circular, semi-circular, elliptical, semi-elliptical, oval, ovoidal, square, rectangular, trapezoidal, triangular and combinations thereof. Shapes having square corners may also be replaced with rounded corners, for example, a rectangle having a square corner replaced by a rounded one may be referred to as a rounded rectangular shape. In one embodiment the cross-sectional shape of the elongated body is selected from cylindrical, rectangular, rounded rectangular, trapezoidal and rounded trapezoidal. In one embodiment the cross-sectional shape of the elongated body is selected from cylindrical, rectangular, rounded rectangular, trapezoidal and rounded trapezoidal, with cylindrical being particularly preferred.

The cross-sectional shape of the mouthpiece end may be the same or different to the rest of the elongated body. In one embodiment, the mouthpiece is tapered towards the mouthpiece hole. In one embodiment the cross-sectional shape of the mouthpiece hole is adapted to fit a conventional aerosol or nebuliser face mask.

As the inhalable liquid may be self-administered by a patient using the device, the device may optionally comprise a lanyard and a point for attachment thereto for placement around the patient's wrist or neck. Accordingly, in one embodiment the device comprises a lanyard and a point for attachment thereto.

### Example 1

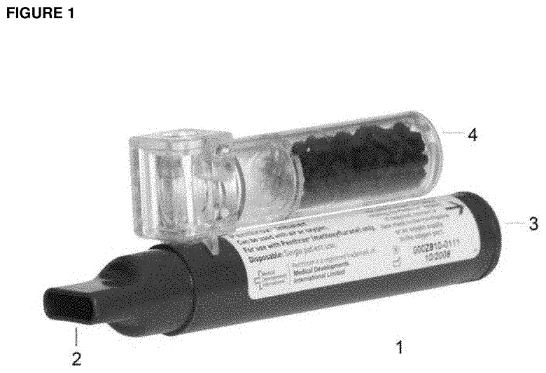

FIG. 1 shows the prior art Green Whistle™ inhaler device (1) (Medical Developments International Limited) which is currently used in Australia for the delivery of Penthrox®/™ (methoxyflurane) as an analgesic (1.5 mL or 3 mL, storage brown glass vial container with screw cap). When required for use, the delivery dose of methoxyflurane is poured into the base end (3) of the device. After the dose is poured into the base end for delivery onto the evaporative means (not shown), the methoxyflurane evaporates so that the patient can self-administer the analgesic by inhaling the air/vapour mix through the mouthpiece (2). Provided that the patient continues to breathe through the mouthpiece, any exhaled air/vapour mix will exit the device via the externally fitted chamber containing activated carbon ‘AC-chamber’ (4).

### Example 2

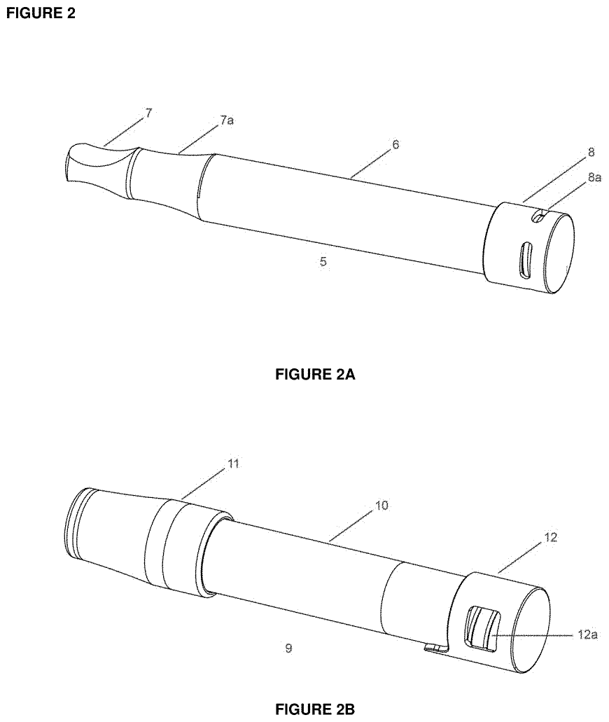

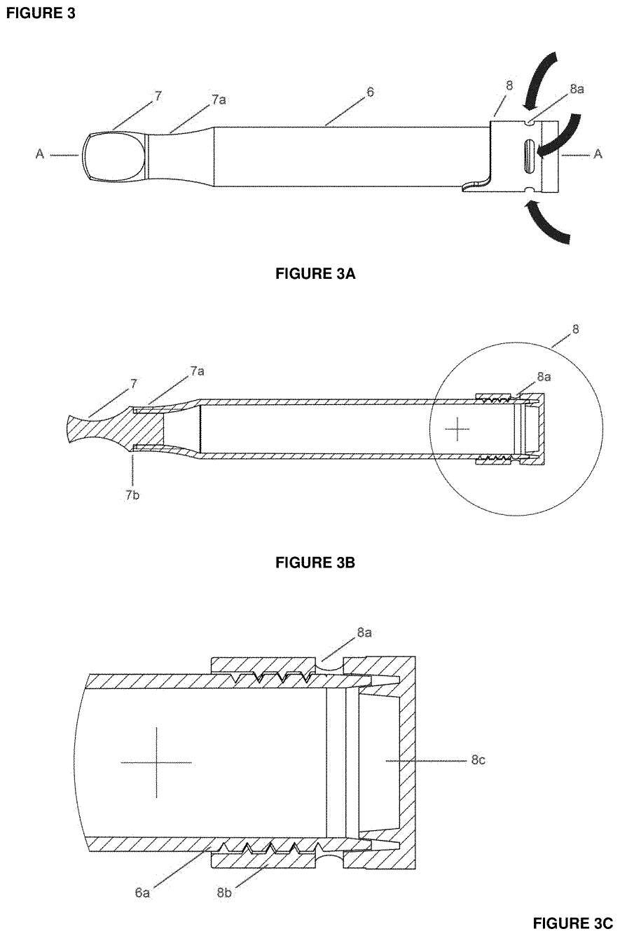

FIG. 2A shows an inhaler device (5) according to an embodiment of the invention. FIG. 3A shows an alternative perspective of the device when viewed from above. The device also comprises an internally stored passive evaporation support material (not shown) which is pre-loaded with an inhalable liquid such as methoxyflurane. Although the passive evaporation support material pre-loaded with the inhalable liquid has not been shown it will be understood to be present for delivery of the air/vapour mix upon inhalation by the patient. In storage mode, the inhaler device functions as a sealed storage container for the inhalable liquid and its vapour so that it is primed and ready for immediate delivery of the drug in vapour form to the patient upon opening. The inhaler device has a sealed elongated body (6), a first end which is adapted to function as a mouthpiece end (7a) and sealed by a resealable end-seal (7), and a second end adapted to function as an air inlet end and sealed by a resealable end-seal in the form of an adjustable end cap (8) comprising one or more air inlet holes (8a). In FIG. 2A, the air inlet hole(s) (8a) are shown in their closed or ‘sealed’ position whereas FIG. 3A shows the air inlet hole(s) in their open or ‘activated’ position. In administration mode, the first end-seal (7) and the second end-seal (8) are both opened to allow the air to be drawn into the vapour chamber (not shown) in the direction of the arrows shown in FIG. 3A upon inhalation by the patient through the mouthpiece end (7a). The first end-seal (7) may be a plug which is adapted to seal and reseal the mouthpiece end (7a) by virtue of a tight tolerance fit with the vapour inhalation opening (7b) and is removable by the patient or administrator by pulling outwardly in a longitudinal direction. Alternatively, the first end-seal (7) may be a plug which is adapted to seal and reseal the mouthpiece end (7a) by virtue of a screw thread which matingly engages with the mouthpiece end (7a) and is removable by the patient or administrator unscrewing the plug to open the vapour inhalation opening (7b). FIG. 3B is a cross-sectional view A-A of the device shown in FIG. 3A. The second end-seal (8) is an adjustable end cap as shown in FIG. 3B. An enlarged view of the adjustable end cap is shown in FIG. 3C. The air inlet hole(s) (8a) are shown in their closed or ‘sealed’ position. The adjustable end cap as shown in FIG. 3C comprises a screw thread (8a) which matingly engages with a screw thread (6a) of the elongated body (6). The air inlet hole(s) may therefore be opened or ‘activated’ partially or fully by the patient or administrator unscrewing the adjustable end cap (8) in relation to the elongated body (6). The adjustable end cap (8) may optionally comprise a wad insert (8c) to assist with sealing and resealing the device for storage mode.

### Example 3

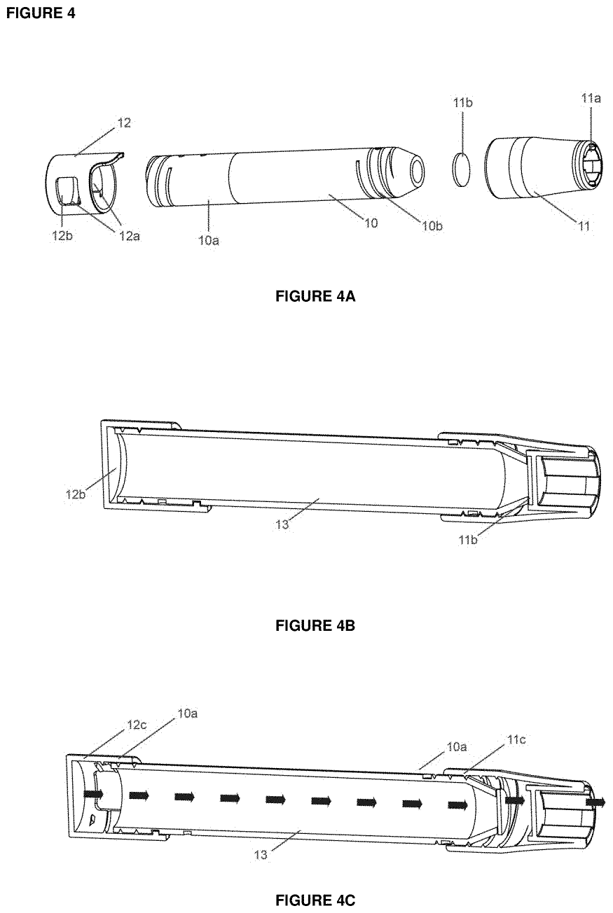

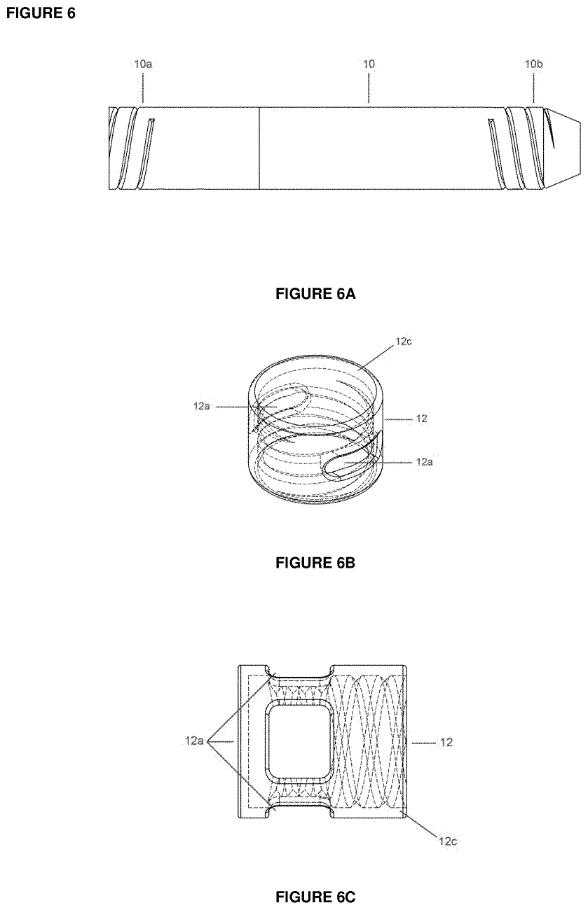

FIG. 2B shows an inhaler device (9) according to an embodiment of the invention. The device comprises a passive evaporation support material (not shown) which is pre-loaded with an inhalable liquid such as methoxyflurane. Although the passive evaporation support material pre-loaded with the inhalable liquid has not been shown it will be understood to be present for delivery of the air/vapour mix upon inhalation by the patient. In storage mode, the inhaler device functions as a sealed storage container for the inhalable liquid and its vapour so that it is primed and ready for immediate delivery of the drug in vapour form to the patient upon opening. The inhaler device has a sealed elongated body (10), a first end which is adapted to function as a mouthpiece end and is sealed by a first end-seal (11) in the form of an adjustable end cap and a second end which is adapted to function as an air inlet end and is sealed by a second end-seal (12) in the form of an adjustable end cap and therefore comprises one or more air inlet holes (12a). An alternative adjustable air inlet end cap (12) with four instead of two air inlet holes (12a) is shown in FIG. 6C. FIG. 4A shows an exploded view of the device to better illustrate the individual components, in particular screw threads (10a) and (10b) of the elongated body (10), the air inlet hole(s) (12a) of adjustable air inlet end cap (12), the vapour inhalation opening (11a) of the adjustable mouthpiece end cap (11) and wad insert (11b) to assist with sealing and resealing the device for storage mode.

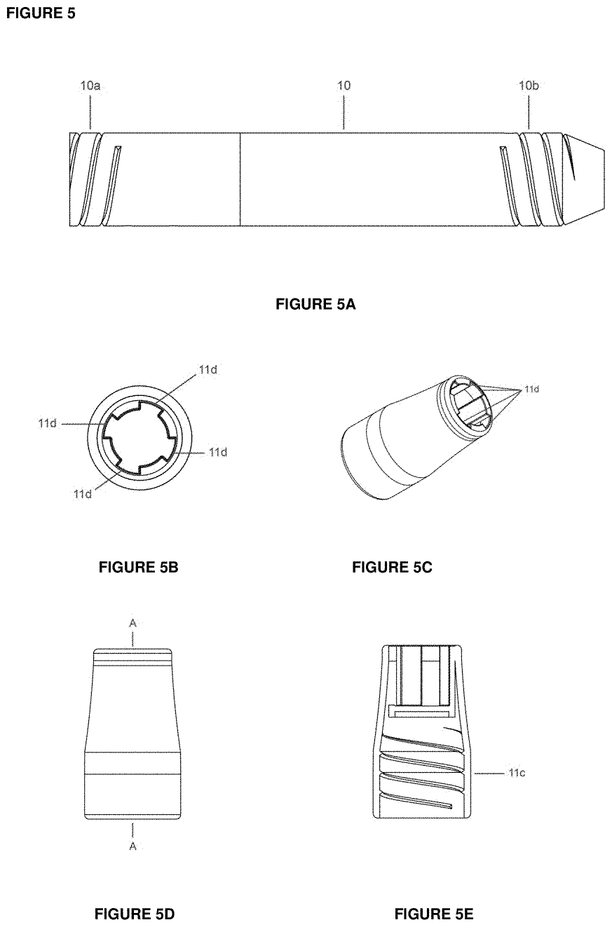

In storage mode, both end-seals are in their closed or ‘sealed’ position as shown in FIG. 4B. In administration mode, both end-seals are in their opened or ‘activated’ position as shown in FIG. 4C. The adjustable air inlet end cap (12) comprises screw thread (12c) to matingly engage with screw thread (10a) and the adjustable mouthpiece end cap (11) comprises screw thread (11c) to matingly engage with screw thread (10b). Screw threads (10a)/(12c) and (10b)/(11c) may be a single screw thread arrangement or alternatively may be double-screw thread arrangement as further illustrated in FIGS. 5A, 5D, 5E, 6A, 6B and 6C. An advantage of a double-screw thread arrangement is to facilitate opening by minimising the number of rotations. Accordingly, the sealed ends are opened by unscrewing end caps (11) and (12) to allow the air to be drawn in through the vapour chamber (13) as shown by the arrows in FIG. 4C upon inhalation by the patient through the mouthpiece end (11). To deliver the air/vapour mix to the patient the adjustable mouthpiece end cap (11) comprises one or more internal grooves (11d) as further illustrated in FIGS. 5B and 5C.

### Example 4

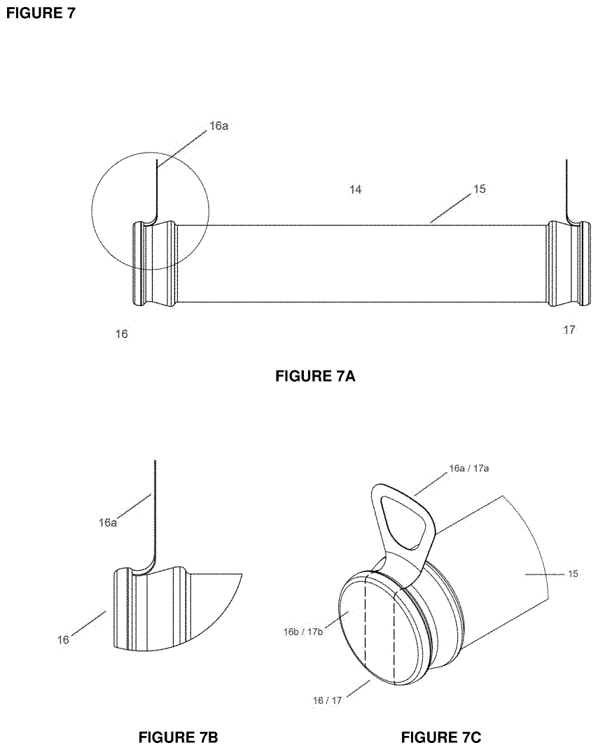

FIG. 7 shows an inhaler device (14) according to an embodiment of the invention. The device comprises a passive evaporation support material (not shown) which is pre-loaded with an inhalable liquid such as methoxyflurane. Although the passive evaporation support material pre-loaded with the inhalable liquid has not been shown it will be understood to be present for delivery of the air/vapour mix upon inhalation by the patient. In storage mode, the inhaler device functions as a sealed storage container for the inhalable liquid and its vapour so that it is primed and ready for immediate delivery of the drug in vapour form to the patient upon opening. As shown in FIG. 7A, the inhaler device has a sealed elongated body (15) sealed by a first non-resealable end-seal (16) and a second non-resealable end-seal (17). The first end-seal (16) and second end-seal (17) are both non-resealable metal ring-pull crown caps and therefore comprise ring pulls (16a) and (17a) respectively. An enlarged view of the non-resealable ring-pull crown cap (16) comprising ring pull (16a) is shown in FIG. 7B. As the first end-seal (16) and the second end-seal (17) are the same and the sealed elongated body has the same cylindrical cross-sectional area throughout the length of the device, either end may function as the mouthpiece end or the air inlet end so that the device may be oriented in either direction for use. When required for use, the non-resealable metal ring-pull crown caps are opened by the administrator/patient pulling ring pulls (16a) and (17a) along ring pull lines (16b) and (17b) respectively as shown in FIG. 7C. The air inlet opening and vapour inhalation opening (not shown) are formed by the circumference of the elongated body (15) and upon removal of end-seals (16) and (17), allow the air to be drawn in through the vapour chamber (not shown) upon inhalation by the patient through whatever end is selected as the mouthpiece end.

### Example 5

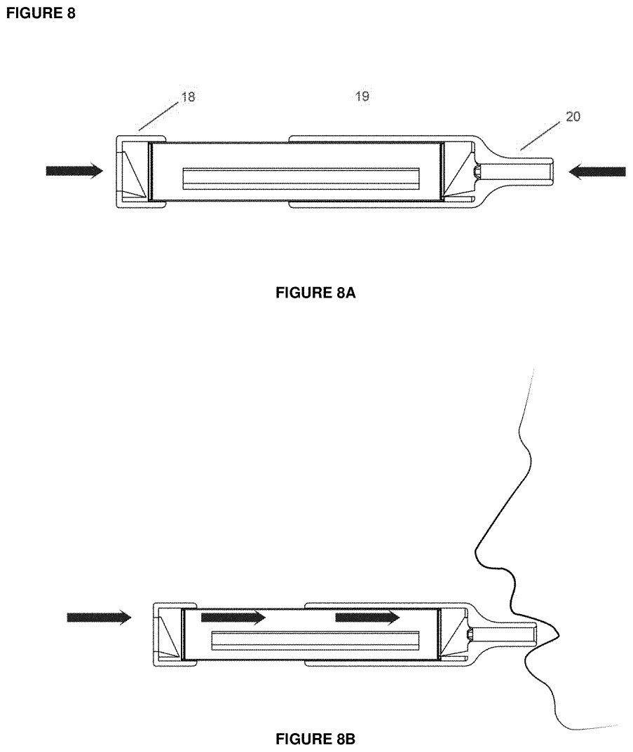

FIG. 8 shows an inhaler device (19) according to an embodiment of the invention. The device is ‘activated’ for use by pushing on the sealed air inlet end (18) and the sealed mouthpiece end (20) in the direction of the arrows shown in FIG. 8A. The patient then inhales through the mouth piece end to administer the air/vapour mix in the direction of the arrows shown in FIG. 8B.

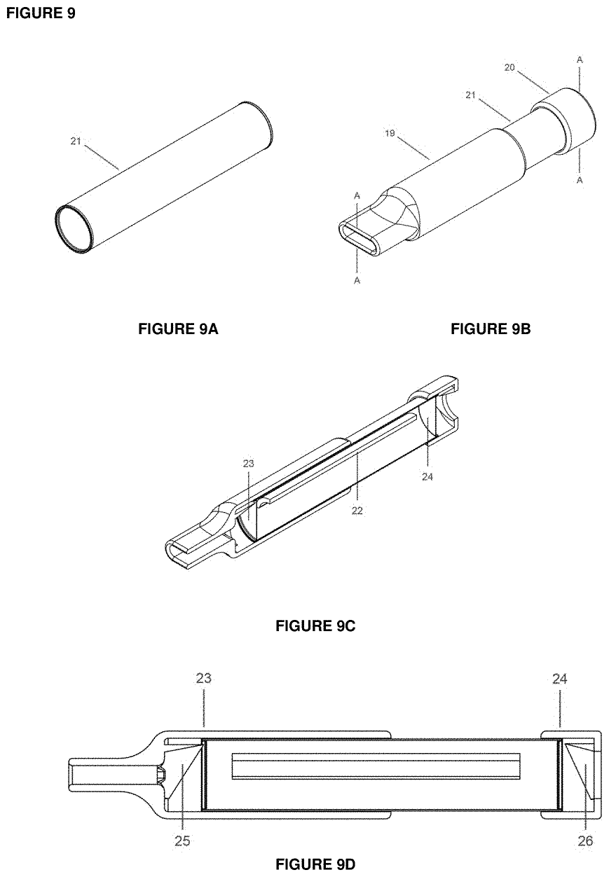

The individual components of the device are further illustrated in FIG. 9. FIG. 9A shows the elongated body (21) in the form of a cylinder. The cylinder may be made of any suitable material although polymeric materials and metals are particularly preferred to withstand the pushing forces required for opening the sealed air inlet end (19) and the sealed mouthpiece end (20). FIGS. 9C and 9D provide cross-sectional views A-A of the assembled device as shown in FIG. 9B. The device comprises a passive evaporation support material (22) as shown in FIG. 9C which is pre-loaded with an inhalable liquid such as methoxyflurane. The passive evaporation support material pre-loaded with the inhalable liquid allows for delivery of the air/vapour mix upon inhalation by the patient once the device is ‘activated’ for use by puncturing the non-resealable end-seals (23) and (24) in the first sealed (mouthpiece) end (20) and second sealed (air inlet) end (19) respectively. To puncture the non-resealable seals (23) and (24), the mouthpiece end and the air inlet end independently comprise a puncturing means (25) and (26) respectively as shown in FIG. 9D.

### Example 6

The ability of an inhaler device to delivery methoxyflurane may be tested using a breath simulator system such as a pulmonary waveform generator system as follows. The pulmonary waveform generator is set to “Adult” flow conditions (14 breaths per minute) and the concentration logging software and Datex Sensor commenced. For each test, methoxyflurane (3 mL) is poured into the device so that the polypropylene wick is pre-loaded with the methoxyflurane to be delivered and the mouthpiece end of the device then inserted into the opening of the pulmonary waveform generator. Concentration logging is then commenced for the first minute for the first breaths concentration and then for the next 20 minutes for steady state testing.

A prototype device for testing may be manufactured as a rapid prototype using a HDPE equivalent material.

Throughout this specification and the claims which follow, unless the context requires otherwise, the word “comprise” and variations thereof such as “comprises” and “comprising”, will be understood to imply the inclusion of a stated integer or step or group of integers or steps but not to the exclusion of any other integer or step or group of integers or steps.

The reference in this specification to any prior publication or information derived from it, or to any matter which is known is not and should not be taken as an acknowledgement or admission or any form of suggestion that prior publication, or information derived from it, or known matter, forms part of the common general knowledge in the field of endeavour to which this specification relates.

1. An inhaler device housing methoxyflurane liquid for delivery to a patient, the inhaler device comprising:
(1) a sealed elongated body having an air inlet end and a mouthpiece end;
(2) a first end-seal sealing the air inlet end of the elongated body;
(3) a second end-seal sealing the mouthpiece end of the elongated body; and
(4) a passive evaporation support material pre-loaded with methoxyflurane liquid;
wherein as the methoxyflurane liquid forms a vapor upon storage, the elongated body forms a vapor chamber such that the stored vapor is available for direct administration to a patient upon opening the first and second end-seals,
wherein the first end-seal is a resealable, adjustable end cap further comprising at least one air inlet groove or hole, said air inlet groove or hole adapted to form an air inlet opening when the resealable, adjustable end cap is opened such that the size of the air inlet is adjustable, and
wherein the second end-seal is a resealable, adjustable end cap configured for insertion into the mouth of a patient, the second end-seal further comprising a mouthpiece chamber configured such that:
when the second end-seal is closed, the vapor chamber is sealed from the mouthpiece chamber; and
when the second end-seal is open, vapor flows radially inward into the mouthpiece chamber through vents located in a side wall of the mouthpiece chamber.

2. The inhaler device according to claim 1 wherein the first end cap is detachably fastened to rotatingly engage with the elongated body of the device.

3. The inhaler device according to claim 2 wherein the first end cap is configured to be rotated in a first direction to close the air inlet opening and in a second direction to open the air inlet opening.

4. The inhaler device according to claim 1 wherein the second end cap has an outer shape that tapers toward an inhalation opening of the mouthpiece chamber.

5. The inhaler device according to claim 1 wherein the second end cap is detachably fastened to rotatingly engage with the elongated body of the device.

6. The inhaler device according to claim 5 wherein the second end cap is configured to be rotated in a first direction to seal the vapor inhalation opening and in a second direction to open the vapor inhalation opening.

7. The inhaler device according to claim 1 wherein second end cap comprises a wad insert to seal the vapor inhalation opening when the mouthpiece end is closed

8. The inhaler device according to claim 7 wherein the wad insert comprises a compressible material and a vapor impermeable film or foil configured to provide a tight seal when the mouthpiece end is closed.

9. The inhaler device according to claim 1 wherein the passive evaporation support material is a polypropylene wicking felt.

10. The inhaler device according to claim 1 wherein the passive evaporation support material comprises three or more radial arms extending from a central portion to an internal surface of the vapor chamber to form three or more longitudinal conduits.

11. The inhaler device according to claim 1, wherein the device consists only of: the sealed elongated body; the first end-seal; the second end-seal; and the passive evaporation support material pre-loaded with the methoxyflurane liquid.
